(19) United States
(12) Patent Application Publication
(10) Pub. No.: US 2007/0083237 A1
(43) Pub. Date: Apr. 12, 2007

(54) Magnetic therapeutic device and method of using the same

(76) Inventor: Elberto Berdut Teruel, San Juan, PR (US)

Correspondence Address: Hector M. Reyes Rivera Ph.D JD, P O Box 190086 hato Rey Station, San Juan, PR, 00919, US

(21) Appl. No.: 11/248,067

(22) Filed: Oct. 12, 2005

(51) Int. Cl. A61N 1/39

(52) U.S. Cl. 607/1

(57) ABSTRACT

A magnetic therapeutic device useful for medical purposes wherein the head of the user is exposes to magnetic elements having opposite polarities simultaneously: one side of the head receive magnetic flux having a north to south polarity at the same time that the opposite side of the other side of the wearer's head is exposed to a south to north polarity. The magnetic therapeutic device comprises two magnetic elements, means for mounting the said magnetic elements and means for holding the said magnetic elements at a particular head location at substantially opposite side to each other. The magnetic therapeutic device is effective in the prevention and/or treatment of medical conditions such as migraine, body pain, sleeplessness, menstrual pain, depression and other stress related conditions or diseases.

### FIELD OF THE INVENTION

This invention relates to a magnetic therapeutic device useful for therapeutic purposes. More particularly, this invention relates to a head magnetic device and a process to treat and or prevent migraine, body pain, sleeplessness, menstrual pain, depression and other stress related conditions or diseases comprising the said magnetic therapeutic device.

### BACKGROUND OF THE INVENTION

The prior art discloses multiple documents wherein a variety of devices or items containing magnets are described and recommended for treating, preventing or improving diverse health conditions.

For instance, Treskov et al, U.S. Pat. No. 6,332,469 discloses a body part shaving items comprising magnets in order to reduce the skin irritation, promoting faster healing of skin cuts and having a prophylactic and healing effect on the body part subjected to shaving. Similarly, Yang, U.S. Pat. No. 5,891,331 discloses a portable magnetic device similar to a fountain pen and useful in purifying water and other drinks by exposing the said liquids to bio ceramics and magnets. The said magnetized liquids are said to promote blood circulation as well as to improve digestion and absorption.

Shimiyashu, U.S. Pat. No. 4,480,596; Smith et al, U.S. Pat. No. 6,623,419 as well as Martello U.S. Pat. No. 6,551,234 and Getek, U.S. Pat. No. 6,406,418 are examples of US patents wherein therapeutic magnetic belts are used in order to reduce musculoskeletal pain, counteract fat and or reducing muscles stiffness by exposing the described body areas to magnetic flux.

Similarly, Roberts et al U.S. Pat. No. 6,632,168 discloses a magnetic therapeutic device and a method to treat pain or injury areas comprising the use of the said magnetic device under the wearer's skin. On the other hand Steponovich, U.S. Pat. No. 6,610,023, discloses a strap having magnets that is conveniently wrapped around the knee, giving support to the knee joint that allows the wearer to have maximum knee activity while helping him or her to overcome pain and diseases associated with the knee.

Similarly everyday use articles such as pads, adhesive bands, patches, bandages and wrapping materials comprising magnetic items and directed to the magnetotherapy of different parts of the body are disclosed in multiple US patents such as Juster et al, U.S. Pat. No. 6,344,021, Engel U.S. Pat. No. 6,146,324, Nagler U.S. Pat. No. 6,093,143, Snider U.S. Pat. No. 5,336,498, Russell U.S. Pat. No. 5,782,743 and Griffin et al U.S. Pat. No. 4,587,956 among others.

Furthermore, magnetic body ornaments and/or jewelry, such as rings, bracelets, necklaces, chain belts, earrings and wrist watches have been already disclosed in patented publications, such as Chiu U.S. Pat. No. 5,989,178, Ishikawa U.S. Pat. No. 4,095,587, Monden et al, U.S. Pat. No. 4,186,567 and Li et al U.S. Pat. No. 5,226,020, among others. The said items are said to promote health conditions in the wearer and in many instances preventing the wearer of getting ill.

The prior art also discloses other multiple magnetic items such as shoes, pillows and gloves that are intended to promote health via exposing the body to the magnetic flux integrated in the said items, see for instance, Qui et al, U.S. Pat. No. 6,151,807, Greenwalt U.S. Pat. No. 5,976,100, Komuro U.S. Pat. No. 5,193,236 and Chen U.S. Pat. No. 6,085,355, among others.

Interestingly, some magneto-therapeutic devices are designed to treat particular medical conditions, for instance, Haglund U.S. Pat. No. 6,053,859 discloses a magnetic apparatus and method comprising the same in the treatment of sinus conditions. On the other hand, Paturu US Patent Application publication 20020151759 discloses a method for treatment of erectile dysfunction, peripheral vascular disease, cerebral insufficiency and certain vascular pathologies while Ross U.S. Pat. No. 5,718,721 and Fichell et al U.S. Pat. No. 6,402,678 disclose a method for the treating of migraine headache pain by exposing selective parts of the body to magnetic flux. Similarly, Wascher et al U.S. Pat. No. 6,443,882 and Bouldin et al U.S. Pat. No. 6,149,577 disclose an apparatus and method comprising the said magneto therapeutic device in the treatment of degenerative diseases and disorders. The full contents of the above cited references are herein incorporated by reference.

In general terms, the prior art recognizes that the magneto therapy functions in the treatment of multiple medical diseases and/or conditions by increasing the blood circulation and energizing a particular part of the body once it is exposed to the magnetic energy or flux of magnets. Increasing blood's circulation helps to oxygenate the vital organs and tissues and help to clear up veins and arteries, stimulating the body to promote its healing function naturally.

Thus there is a need for new methods of using magneto therapeutic devices in treating medical conditions and/or diseases such as migraine, muscle pain, sleeplessness, menstrual pain, stress related conditions and/or diseases, wherein the said method is a noninvasive technique that does not requires the use of medical compositions and lacks the risk of secondary effects.

### SUMMARY OF THE INVENTION

An object of the instant condition is to provide a non invasive apparatus useful in the magneto-therapy field and able to heal and or treat a variety of medical conditions such as migraine, body pain, sleeplessness, menstrual pain, depression and other stress related diseases or conditions.

Another object of the instant invention is to provide a method of healing or treating medical conditions such as headaches, migraine, pain, depression and stress related conditions by using a magnetic therapeutic device. Another further object of the instant invention is to provide a prophylactic method of avoiding health problems; improving health conditions and general blood circulation by using a novel magneto therapy device and wherein at least two magnets are used, and particularly wherein the magnets are placed with a polarity of north to south on one side of the wearer's head while at the opposite side a the other magnet is placed south to north in such a way that one side of the wearer 's head is exposed to a north to south magnetic flux while the other side is exposed south to north.

The phrase stress related conditions or diseases as herein used is defined as those medical conditions or diseases wherein stress, even though is not the sole cause of the said disease or condition, it plays or it is known to play an important role in the initiation, maintenance, perpetuation and/or exacerbation of the symptoms. Particular examples of stress related conditions or diseases are High Blood Pressure, Ischemic Heart Disease, Peptic Ulcer, Irritable Bowel Syndrome, Asthma, Tension Headache, Psychoneuroses, Fatigue, Insomnia, Sexual Dysfunction, Alcoholism, Smoking and Skin diseases such as Psoriasis, Atopic Dermatitis, Neurodermatitis and the like.

### BRIEF DESCRIPTION OF THE DRAWINGS

FIG. 1 illustrates a top view of a wearer's head using the therapeutic magnetic device.

FIG. 2 Illustrates front view of a user wearer using the therapeutic magnetic device.

FIG. 3 Illustrates side views of embodiments wherein the said magnets are positioned in caps, helmet and hats of different styles or headphone like structures having the magnetic elements at the extreme parts of the said structure.

FIG. 4 Illustrates a perspective view of the magnetic element already mounted in a plate.

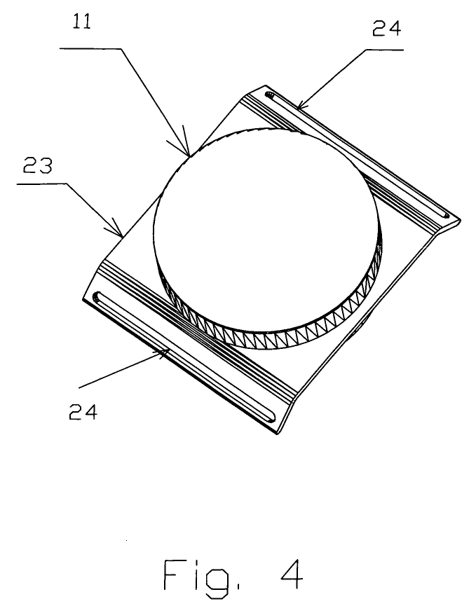

FIG. 5 Illustrates the embodiment wherein the magnetic elements are mounted in a plate and further inserted in a belt or strap.

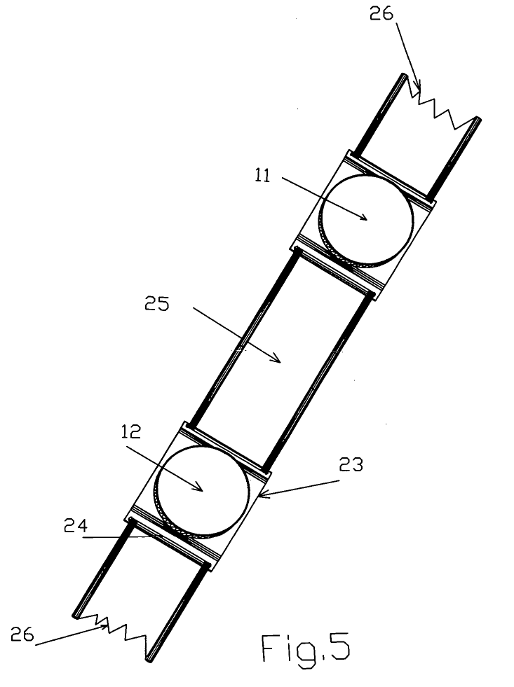

FIG. 6 Illustrates examples of geometrical forms of magnets that may be used in the instant magnetic therapeutic device.

### DETAILED DESCRIPTION OF THE PREFERRED EMBODIMENTS

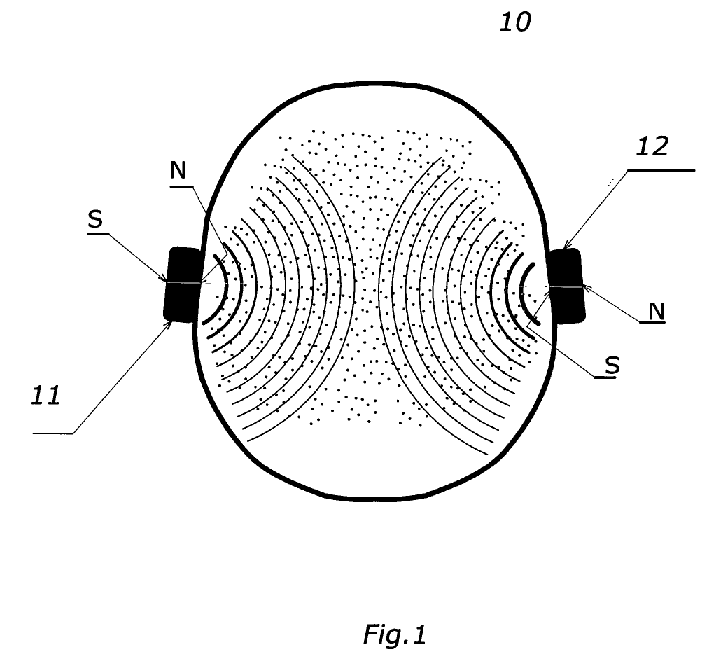

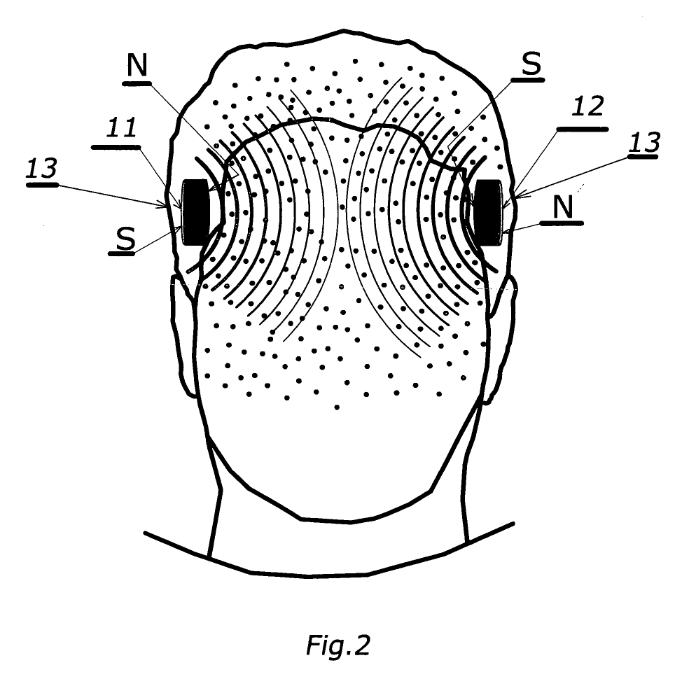

The novel magnetic therapeutic device 10 comprises two magnet elements 11 and 12, wherein each one of the said magnet elements are positioned at a substantially aligned position across the wearer's head as illustrated in FIGS. 1 and 2. Notice that the said magnetic elements are oriented in such a way that one of them has a polarity directed south to north (11) , while the other has its polarity oriented in the opposite way, north to south (12), in such a way that the magnetic flux emitted by the magnets an directed to the head are opposite to each other. Regardless of the relative position-left or right—in the wearer's head, the magnetic elements 11 and 12 should be oriented with an opposite polarity relative to each other. In another words, one of the magnetic element's polarity should be oriented south to north while the other should be oriented north to south irrespectively of the head side-left or right—wherein anyone of the particular magnetic elements is located.

FIG. 2 illustrates the particular place of the head 13 wherein the magnetic elements should preferably be placed: substantially the region between the forehead bone and the ears.

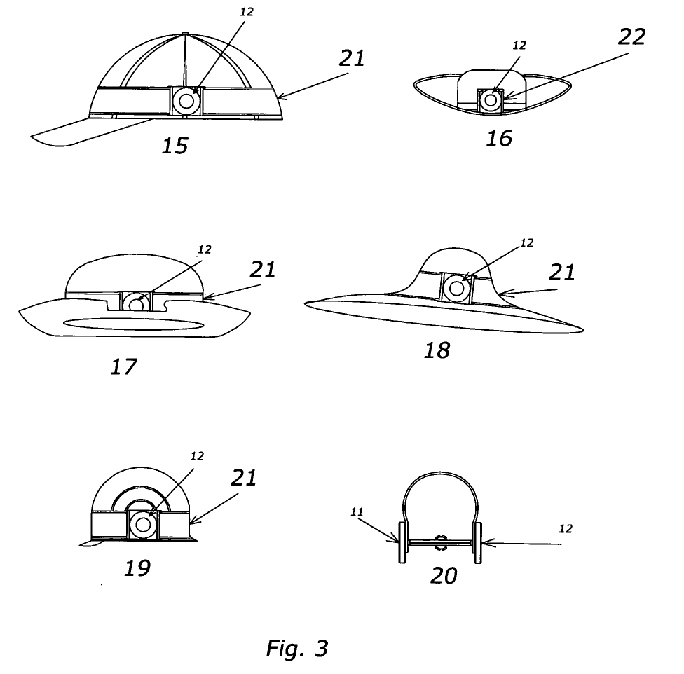

It should be noticed that the means to hold or mount the magnetic elements while important, since it provides the proper fixing and stability to place the magnet elements 11 and 12, are not essential to the device's functioning. Therefore, the said magnetic elements may be mounted and or held by any conventional means for mounting or holding different elements to be wore around the head area, as long as the proper fixing and stability are provided by the selected means. For instance, as illustrated in FIG. 3, the said magnetic elements 11 or 12 may be placed in caps 15 and 16 or hats 17 and 18, or even construction helmet as the one illustrated in 19. The said cap 15 or hats 17 and 18 may even incorporate belts (21) wherein the magnetic elements 11 or 12 are mounted and may be tightened up to the convenience of the wearer in such way that the proper fixing and tightness required is achieved. Alternatively, the said magnetic elements 11 or 12 may be inserted in lateral pockets (22) as illustrated in the lateral sides of hat 16.

Similarly, the same principles for mounting and holding the magnetic elements may be used in headphone-like structures such like (20), wherein the said magnet containing pockets or magnets mounted in plates or similar ones are assembled or connected at the extremes of the said headphone structure (20) or the like.

Alternatively, the magnetic elements 11 and 12 may be mounted in the particular mounting plate (23), preferably wherein the said plate is metallic and even more preferably made of stainless steel or similar metals. The said plate may be planar and rectangular and wherein the two shortest sides of the said rectangular plate having an inclined angle or curve of substantially 45 degrees, and further having a slot (24) in the said inclined area. A belt or strap 25 may be inserted through slots 24 in order to slide the mounting plate (23) having the magnetic elements 11 or 12, thus the belt 25 becoming the means for holding the mounting plate 23. The said belt 25 is preferably made of a strong, flexible non-elastic material, such as leather, plastic, fabrics or combinations thereof. The said belt or strap 25 should also have a mechanism for fastening preferably at ending extremes 26. The said fastening mechanism may be any conventional fastening mechanism such as buckle like or a male-female adaptor, among many others.

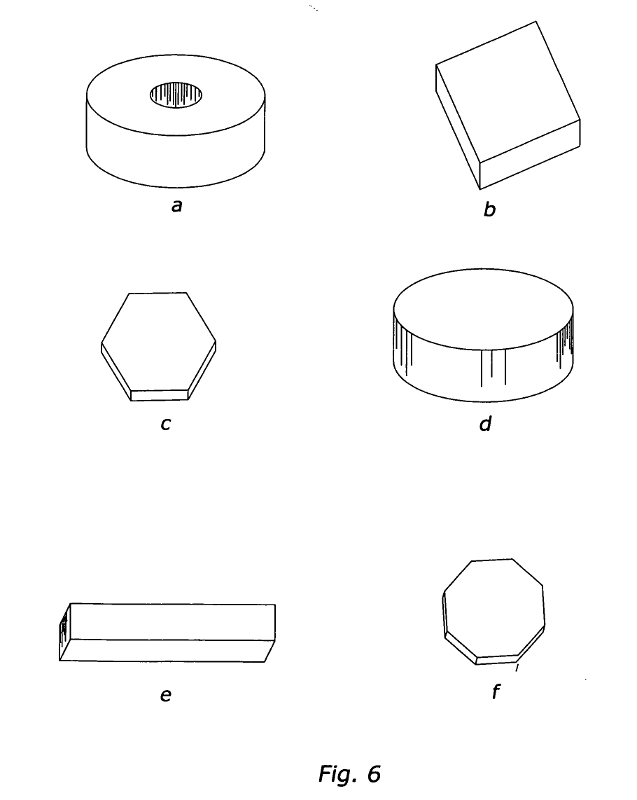

The required magnets in the magnetic elements 11 and 12 may contain variable magnetic strength and shapes. Some of the possible shapes, among others are illustrated in FIG. 6, forms a through f.

The term magnetic element herein used is intended to describe the part of the device containing one or more magnets. If more than one magnet is used in a given magnetic element, they may be placed in a stacked configuration. The phrase stacked configurations is intended to mean herein that a magnet is magnetically placed over the top of the other successively, thus each stack of magnets forming what is herein called a magnetic element unit. Optionally, magnets can also be glued to one another even when they are magnetically attractive to each other. In case of the embodiment wherein the plate is used, a magnet may be placed at the bottom surface of the said plate while one or more can be placed on the top surface. Whenever the said plate is not magnetically attractive to the magnets placed over it, they may be glued to the said surface.

The said magnets may be formed by any suitable magnetic material, preferably from rare earth, neodymium, ceramic or mixture thereof. The intensity of the magnetic force of the said magnets can be variable and easily increased or decreased by the user by simply adding or subtracting magnets from the device at the wearer's convenience.

### METHOD OF USING THE DEVICE

The herein described device may be used in the treatment of headache, migraine, pain insomnia and stressfulness related conditions or in the prevention or prophylactic measure of the said conditions. The said prevention or treatment is achieved by placing the magnetic elements to the head so that the said magnetic elements are positioned in the sides of the head substantially located between the forehead and the ears as illustrated in FIGS. 1 and 2 or in nearby areas. The said magnetic elements should be placed in a substantially aligned position. The device should be used by a suitable time, depending on the response of the wearer's body. Moreover, the device may be used as a prophylactic measure or when suffering any pain, tension or stressfulness condition. The device is helpful in decreasing or eliminating pain, lack of sleep or stress related conditions.

The above description are those of the preferred embodiments on the instant invention and it is understood that various alterations and or changes made be made without departing from the spirit and broader aspects of the invention as defined in the appended claims, which are to be interpreted in accordance with the principles of patent law including the doctrine of equivalents.

What is claimed is:

1) A magnetic therapeutic device useful in the treatment of headaches, migraine, insomnia, depression, pain and stress related conditions and/or diseases as well as in the improvement of wearer's health comprising:
a) two magnetic elements wherein each one of the said magnetic elements may be positioned at a substantially aligned position to each other and wherein the said magnetic elements contain one or more magnets having variable magnet strength and wherein one of the said magnetic elements has a polarity north to south while the other magnetic element has a south to north polarity;
b) means for mounting the magnetic elements and;
c) means for holding the two mounted magnetic elements so once the said magnetic elements are mounted, they may be adjustable at a substantially aligned position to each other.

2) The magnetic therapeutic device of claim 1, wherein the means for holding the magnetic elements comprises a headphone like structure having the said mounted magnetic elements located at the extremes of the said headphone like structure.

3) The magnetic therapeutic device of claim 1, wherein the means for holding the magnetic elements comprises a sport like cap or hat having a pocket-like structure at left side and right side of the said cap or hat, wherein the said pockets are the means for mounting the magnets and wherein the magnetic elements are positioned in the said pockets.

4) The magnetic therapeutic device of claim 1, wherein the means for holding the magnetic elements comprises a sport like cap or hat having a belt having mounting plates at left side and right side of the said cap or hat, wherein the said plates are the means for mounting the magnets and wherein the magnetic elements are positioned in the said pockets.

5) The magnetic therapeutic device of claim 1, wherein the means for holding the magnetic elements comprises a belt or strap having two sliding metallic plates, wherein the said magnetic elements are mounted and wherein each one of the said metallic plates have two slots at substantially opposite sides of the said plate; from wherein the said belt is inserted in and wherein by sliding the said metallic plates the mounted magnetic elements can be positioned in a substantially opposite position across the wearer's head and wherein the said belt or strap contains fastening elements to fix the said belt or strap to the wearer's head at his or her preference.

6) The magnetic therapeutic device of claim 1, wherein the magnets are selected from a group consisting of rare earth, neodymium, ceramic materials or mixtures thereof.

7) The magnetic therapeutic device of claim 1, wherein the magnets are neodymium magnets.

8) The magnetic therapeutic device of claim 6, wherein the magnets are ceramic magnets.

9) The magnetic therapeutic device of claim 1, wherein the magnets have a cylinder, annular, hexagonal, octagonal or square form.

10) A method of treating headache, migraine, pain, depression, insomnia and stress related diseases or conditions using the magnetic device of claim 1, the said method comprising the steps of:
a) placing the magnetic therapeutic device around the head so that one of the magnetic elements has a polarity north to south while the other magnetic element has a polarity south to north and
b) adjusting the said device to the wearer's head so that each one of the magnetic elements are positioned substantially around the head area between the forehead and the ears and
c) substantially aligning the magnetic elements to each others
d) maintaining the said aligned and adjusted device around the wearer's head during a suitable time.

11) The method of claim 10 wherein the stress related conditions are high blood pressure, ischemic heart disease, peptic ulcer, irritable bowel syndrome, asthma, tension headache, psychoneuroses, fatigue, insomnia, sexual dysfunction, alcoholism, smoking and skin diseases such as psoriasis, atopic dermatitis, neurodermatitis.

12) The method of claim 10 wherein the stress related conditions are skin diseases selected from the group of psoriasis, atopic dermatitis, neurodermatitis.

13) The method of claim 9 wherein the treatment is directed to migraine.

14) The method of claim 9 wherein the stress related conditions are selected from the group of high blood pressure, ischemic heart disease, peptic ulcer, irritable bowel syndrome, asthma, tension headache, psychoneuroses, fatigue, insomnia, sexual dysfunction, alcoholism, smoking.

15) A method of preventing headache, migraine, pain, depression, insomnia and stress related diseases or conditions using the magnetic device of claim 1, the said method comprising the steps of:
a) placing the magnetic therapeutic device around the head so that one of the magnetic elements has a polarity north to south while the other magnetic element has a polarity south to north;
b) adjusting the said device to the wearer's head so that each one of the magnetic elements are positioned substantially around the head area between the forehead and the ears;
c) substantially aligning the magnetic elements to each other and
d) maintaining the said aligned and adjusted device around the wearer's head during a suitable time.
